(19) United States
(12) Patent Application Publication
(10) Pub. No.: US 2025/0313654 A1
(43) Pub. Date: Oct. 9, 2025

(54) ANTIOXIDANT DERIVATIVE OF HYALURONIC ACID

(71) Applicant: University of Vermont and State Agricultural College, Burlington, VT (US)

(72) Inventor: Robert J. HONDAL, Burlington, VT (US)

(21) Appl. No.: 18/865,518

(22) PCT Filed: May 15, 2023
(86) PCT No.: PCT/US23/67001

§ 371 Date: Nov. 13, 2024

Related U.S. Application Data
(60) Provisional application No. 63/341,592, filed on May 13, 2022.

(51) Int. Cl. C08B 37/08; A61K 8/73; A61Q 19/00; C07H 7/06

(52) U.S. Cl. CPC C08B 37/0072; A61K 8/735; A61Q 19/00; C07H 7/06

(57) ABSTRACT

Described herein is a polymer and method that enhances the antioxidant function of hyaluronic acid by synthesizing a hyaluronic-amino acid conjugate whereby the antioxidant amino acid 2-thiohistidine is coupled to the polymer via an amide linkage between the carboxylic acid of hyaluronic acid and the amino group of 2-thiohistidine. Also provided are monomeric compounds with 2-thiohistidine. Included in the present disclosure are the following:

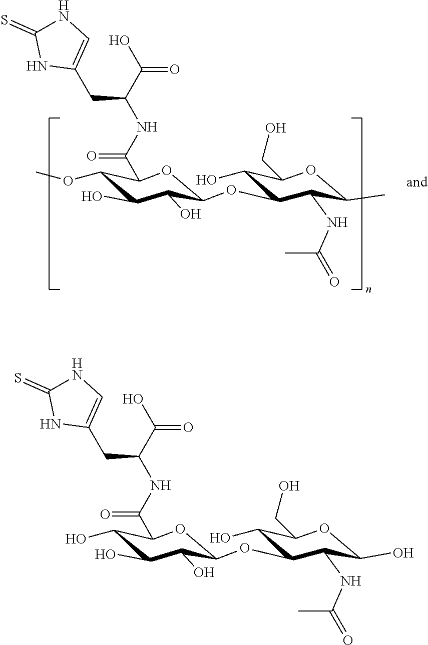

### CROSS REFERENCE TO RELATED APPLICATION

This application claims priority to U.S. Provisional Application No. 63/341,592, filed May 13, 2022, the disclosure of which is incorporated by reference.

### BACKGROUND OF THE DISCLOSURE

Hyaluronic acid is a glycosaminoglycan polymer and a component of the extracellular matrix of the dermis and other tissues. It is a humectant, which means it binds water and as a result, is used in many cosmetics as a moisturizer. Hyaluronic acid has several functions including creating the proper hydrated microenvironment of the extracellular matrix so that nutrients may pass, keratinocyte proliferation, wound healing, and an antioxidant function. It is used as a dermal filler for some types of surgery.

### SUMMARY OF THE DISCLOSURE

Described herein is a polymer and method that greatly enhances the antioxidant function of hyaluronic acid by synthesizing a hyaluronic-amino acid conjugate whereby the novel antioxidant amino acid 2-thiohistidine is coupled to the polymer via an amide linkage between the carboxylic acid of hyaluronic acid and the amino group of 2-thiohistidine as shown in FIG. 1. 2-thiohistidine is an analogue of the antioxidant amino acid ergothioneine, which is produced by fungi and certain bacteria. For humans, it is a candidate vitamin that is obtained in the diet and concentrated in tissues such as bone marrow, monocytes, and various organs. The conjugate of the present disclosure has the advantage of coupling a natural dietary derived antioxidant with a popularly used ingredient of many types of cosmetics to yield a unique cosmeceutical. The conjugate shown in FIG. 1 will function in an identical way to hyaluronic acid that is added to cosmetics with the added advantage that it will be able to quench free radicals as well as singlet oxygen that is produced from exposure to sunlight that is responsible for photoaging. In addition to the antioxidant properties that the newly synthesized conjugate will bring to existing cosmetic products is that once the conjugate reacts with a free radical, it will desulfurize, yielding a sulfate anion and a hyaluronic acid-histidine conjugate as shown in FIG. 2. Histidine is just an amino acid that is found in proteins in human and all other organisms and is non-toxic. The sulfate that is released can be absorbed by the human body and used for the synthesis of sulfated glycosaminoglycans. Sulfate is also a non-toxic by product.

In an aspect, the present disclosure provides polymer compounds. The polymeric compounds comprise a dihydroimidazolethionyl group. The dihydroimidazolethionyl group may be a part of a 2-thiohistidinyl group. The amino acid 2-thiohistindine has the following structure:

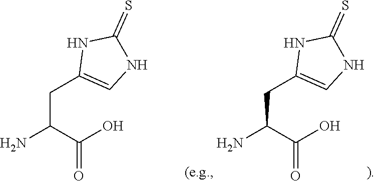 [SMILES: NC(Cc1c[nH]c(=S)[nH]1)C(=O)O ; N[C@@H](Cc1c[nH]c(=S)[nH]1)C(=O)O]

Also provide are compounds comprising one or more dihydroimidazolethionyl groups.

In an aspect, the present disclosure provides compositions. The composition may comprise a polymeric compound and/or compound of the present disclosure. The composition may also comprise a pharmaceutically acceptable carrier.

The composition can comprise a polymeric compound and/or compound in a pharmaceutically acceptable carrier (e.g., carrier). The carrier can be an aqueous carrier suitable for administration to individuals including humans. The carrier can be sterile. The carrier can be a physiological buffer. Examples of suitable carriers include sucrose, dextrose, saline, and/or a pH buffering element (such as, a buffering element that buffers to, for example, a pH from pH 5 to 9, from pH 6 to 8, (e.g., 6.5)) such as histidine, citrate, or phosphate. Additionally, pharmaceutically acceptable carriers may be determined in part by the particular composition being administered. Accordingly, there are a wide variety of suitable formulations of pharmaceutical compositions of the present disclosure. Additional, non-limiting examples of carriers include solutions, suspensions, and emulsions that are dissolved or suspended in a solvent before use, and the like. The composition may comprise one or more diluents. Examples of diluents, include, but are not limited to distilled water, physiological saline, vegetable oil, alcohol, dimethyl sulfoxide, and the like, and combinations thereof. Compositions may contain stabilizers, solubilizers, suspending agents, emulsifiers, soothing agents, buffers, preservatives, and the like, and combinations thereof. Compositions may be sterilized or prepared by sterile procedure. A composition of the disclosure may also be formulated into a sterile solid preparation, for example, by freeze-drying, and may be used after sterilization or dissolution in sterile injectable water or other sterile diluent(s) immediately before use. Additional examples of pharmaceutically acceptable carriers include, but are not limited to, sugars, such as, for example, lactose, glucose, and sucrose; starches, such as, for example, corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as, for example, peanut oil, cottonseed oil, safflower oil, sesame oil, olive oil, corn oil, and soybean oil; glycols, such as, for example, propylene glycol; polyols, such as, for example glycerin, sorbitol, mannitol, and polyethylene glycol; esters, such as, for example, ethyl oleate and ethyl laurate; agar; buffering agents, such as, for example, magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Additional non-limiting examples of pharmaceutically acceptable carriers can be found in: Remington: The Science and Practice of Pharmacy (2012) 22nd Edition, Philadelphia, PA. Lippincott Williams & Wilkins. For example, a composition comprises a modified peptide, and a sterile, suitable carrier for administration to individuals including humans—such as a physiological buffer such as sucrose, dextrose, saline, pH buffering (such as from pH 5 to 9, from pH 7 to 8, from pH 7.2 to 7.6, (e.g., 7.4)) element such as, for example, histidine, citrate, or phosphate. In various examples, the composition may be suitable for injection. Parenteral administration includes infusions and injections, such as, for example, intramuscular, intravenous, intraarterial, intraperitoneal, subcutaneous administration, and the like.

In an aspect, the present disclosure provides methods of using polymeric compounds and/or compounds of the present disclosure of the present disclosure. The method may be a method for quenching free radicals or reducing oxidative stress via reduction of the thiohistindyl group.

### BRIEF DESCRIPTION OF THE FIGURES

For a fuller understanding of the nature and objects of the disclosure, reference should be made to the following detailed description taken in conjunction with the accompanying figures.

FIG. 1 shows a synthesis of proposed hyaluronic acid-2TH conjugate.

FIG. 2 shows a reaction of conjugate with free radical.

### DETAILED DESCRIPTION OF THE DISCLOSURE

Although claimed subject matter will be described in terms of certain embodiments/examples, other embodiments/examples, including embodiments/examples that do not provide all of the benefits and features set forth herein, are also within the scope of this disclosure. Various structural, logical, process and step changes may be made without departing from the scope of the disclosure.

All ranges provided herein include all values that fall within the ranges to the tenth decimal place, unless indicated otherwise.

In this application, the use of the singular form encompasses the plural and vice versa.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent, trivalent, and the like, radicals). Illustrative examples of groups include:

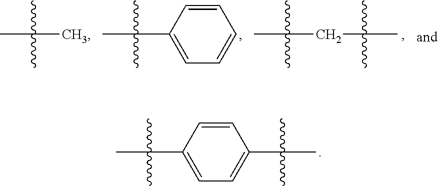

Amino acids may be referred to herein by either their commonly known three letter symbols or by the one-letter symbols recommended by the IUPAC-IUB Biochemical Nomenclature Commission.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. In an example, about refers to ±1%, ±2%, ±3%, ±4%, ±5%, ±6%, ±7%, ±8%, ±9%, ±10%, ±15%, or ±20%.

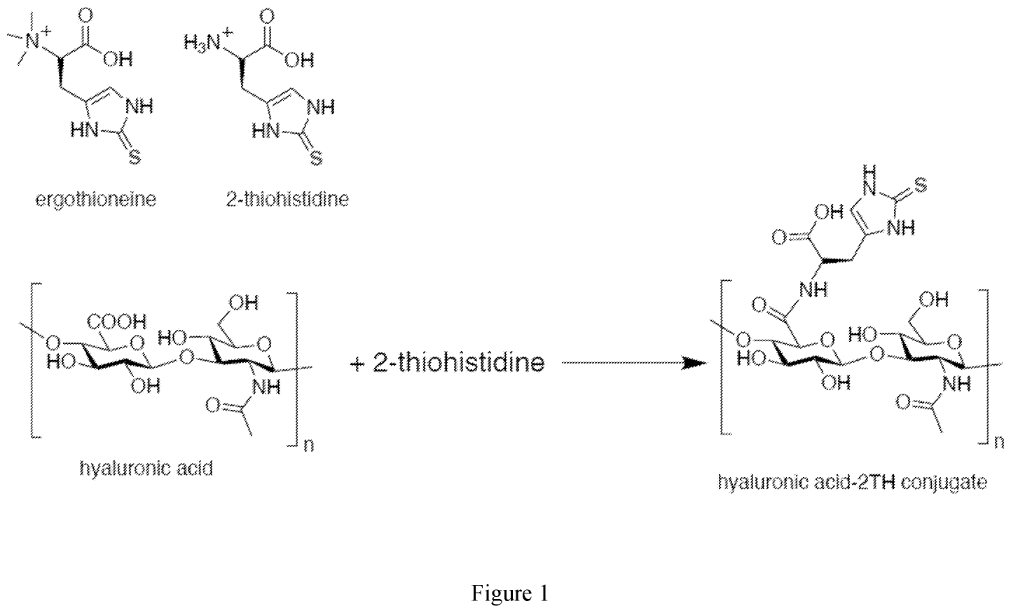

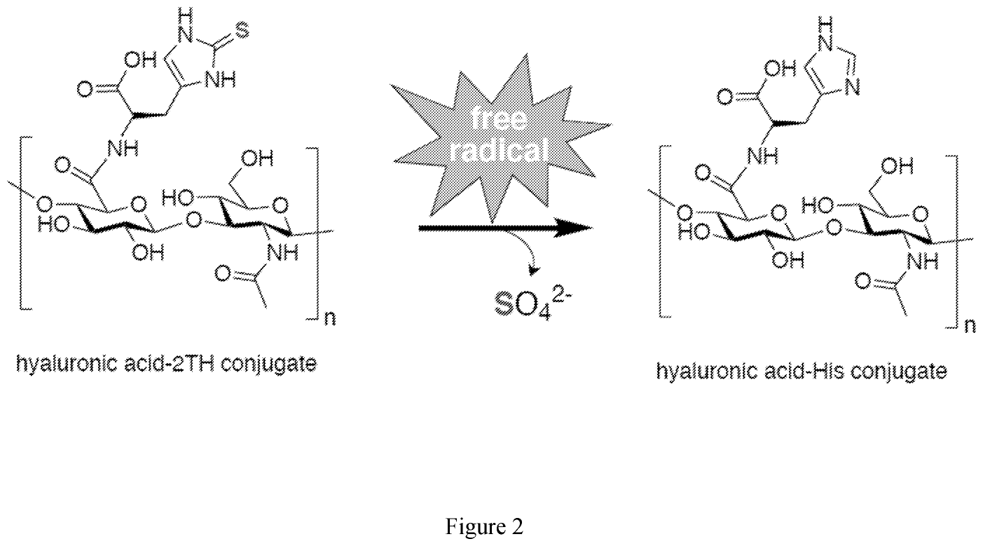

Described herein is a polymer and method that greatly enhances the antioxidant function of hyaluronic acid by synthesizing a hyaluronic-amino acid conjugate whereby the novel antioxidant amino acid 2-thiohistidine is coupled to the polymer via an amide linkage between the carboxylic acid of hyaluronic acid and the amino group of 2-thiohistidine as shown in FIG. 1 below. 2-thiohistidine is an analogue of the antioxidant amino acid ergothioneine, which is produced by fungi and certain bacteria. For humans, it is a candidate vitamin that is obtained in the diet and concentrated in tissues such as bone marrow, monocytes, and various organs. The conjugate of the present disclosure has the advantage of coupling a natural dietary derived antioxidant with a popularly used ingredient of many types of cosmetics to yield a unique cosmeceutical. The conjugate shown in FIG. 1 will function in an identical way to hyaluronic acid that is added to cosmetics with the added advantage that it will be able to quench free radicals as well as singlet oxygen that is produced from exposure to sunlight that is responsible for photoaging. In addition to the antioxidant properties that the newly synthesized conjugate will bring to existing cosmetic products is that once the conjugate reacts with a free radical, it will desulfurize, yielding a sulfate anion and a hyaluronic acid-histidine conjugate as shown in FIG. 2. Histidine is just an amino acid that is found in proteins in human and all other organisms and is non-toxic. The sulfate that is released can be absorbed by the human body and used for the synthesis of sulfated glycosaminoglycans. Sulfate is also a non-toxic by product.

In an aspect, the present disclosure provides polymer compounds. The polymeric compounds comprise a dihydroimidazolethionyl group. The dihydroimidazolethionyl group may be a part of a 2-thiohistidinyl group. The amino acid 2-thiohistindine has the following structure:

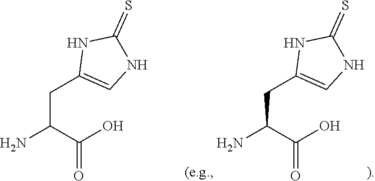 [SMILES: NC(Cc1c[nH]c(=S)[nH]1)C(=O)O ; N[C@@H](Cc1c[nH]c(=S)[nH]1)C(=O)O]

Also provide are compounds comprising one or more dihydroimidazolethionyl groups.

In various examples, a polymeric compound has the following structure:

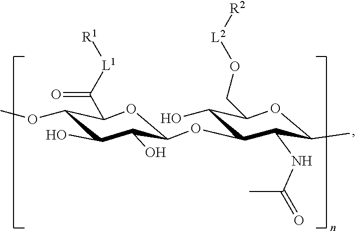

where L¹ and L² are each optional and are linking groups; R¹ is OH or

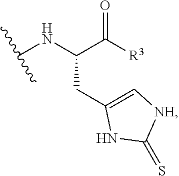

R³ is OH, —OCH₃, L¹, L¹-R³, or

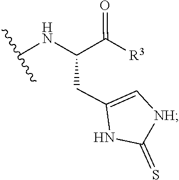

R² is H or

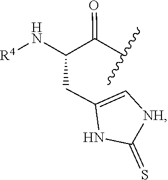

wherein R⁴ is H, L², L²-R⁴, or

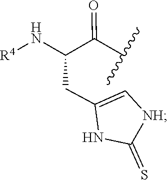

n is 2 to 50, including all integer values and ranges therebetween; and
where at least one of

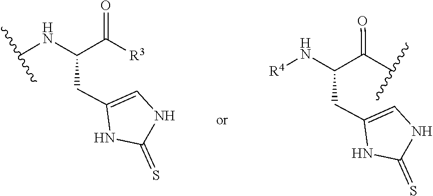

is present. A polymeric compound may have an Mn and/or Mw of 500-10,000 Da. In various embodiments, the polymeric compound is part of a larger polymer (e.g., the structure is incorporated into another polymer, such as, for example, a block co-polymer).

In an example, a compound of the present disclosure has the following structure:

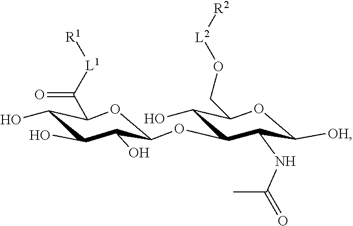

where L¹ and L² are each optional and are linking groups; R¹ is OH or

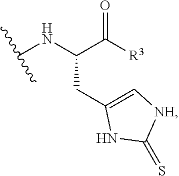

R³ is OH, —OCH₃, L¹, L¹-R³, or

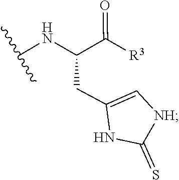

R² is H, L², L²-R², or

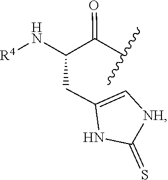

wherein R⁴ is H, L², L²-R⁴, or

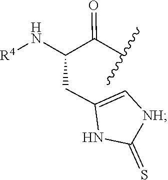

where at least one of

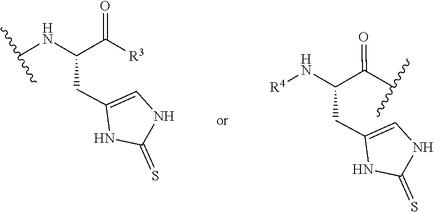

is present.

Various linking groups may be used. Examples of linking groups include, but are not limited to amino acid groups, peptides groups, PEG groups, diethylamine groups, ethyl disulfide groups, cysteamine groups, and the like, and combinations thereof. In various examples, where a linking group is an R³ or R⁴ group, the linking group may be a terminal end group; that is, the linking group may be a monovalent group. For example, non-limiting examples of linking group in such an embodiment include —CH₂CH₂OCH₂CH₂OH, —NHCH₂CO₂H, or —COCH₂NH₂, —OCH₃, or the like. In various examples, the amino acid linking groups are glycyl groups or prolyl group.

In various embodiments, the polymeric compound has the following structure:

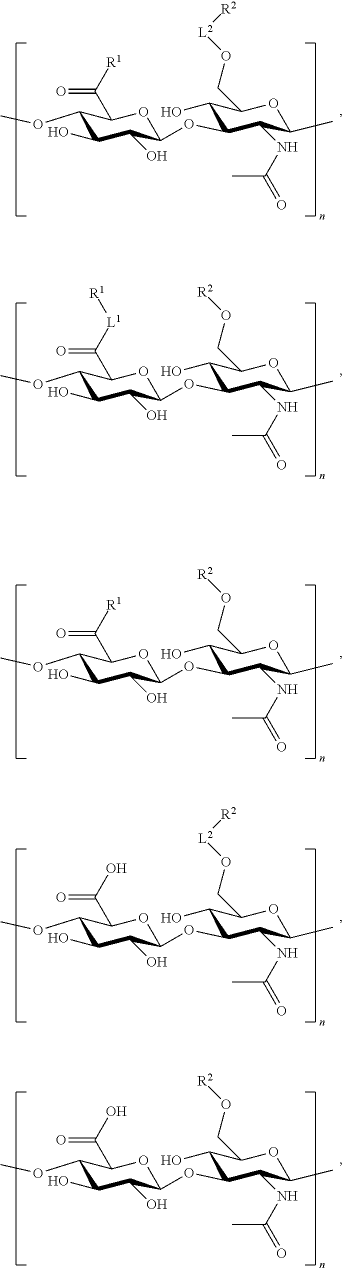

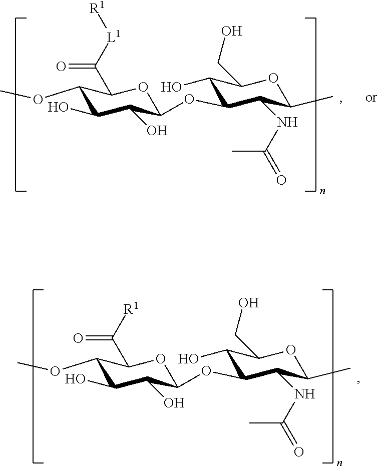

where are least one of

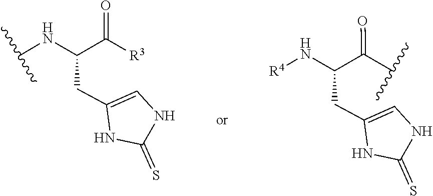

is present. In various examples where a polymeric compound has an R¹, at least a portion of the R¹ groups (e.g., at least one) are

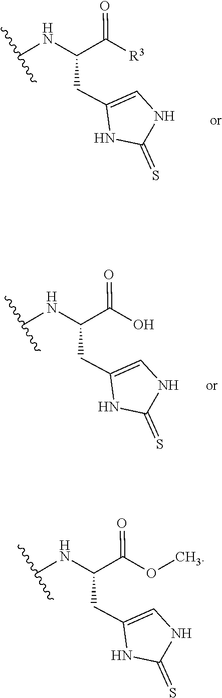

In various examples, the polymeric compound has the following structure:

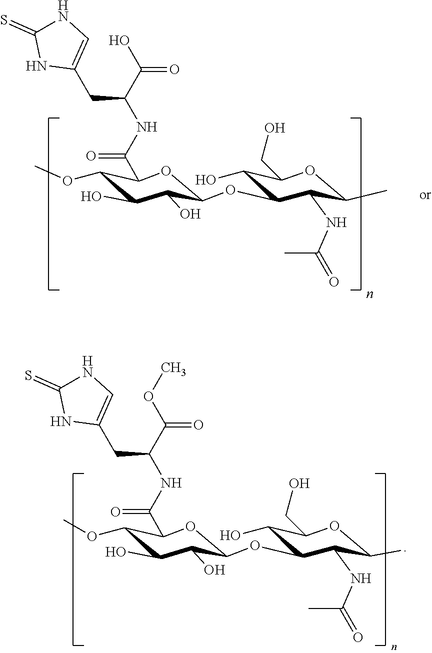

In various embodiments, the compound has the following structure:

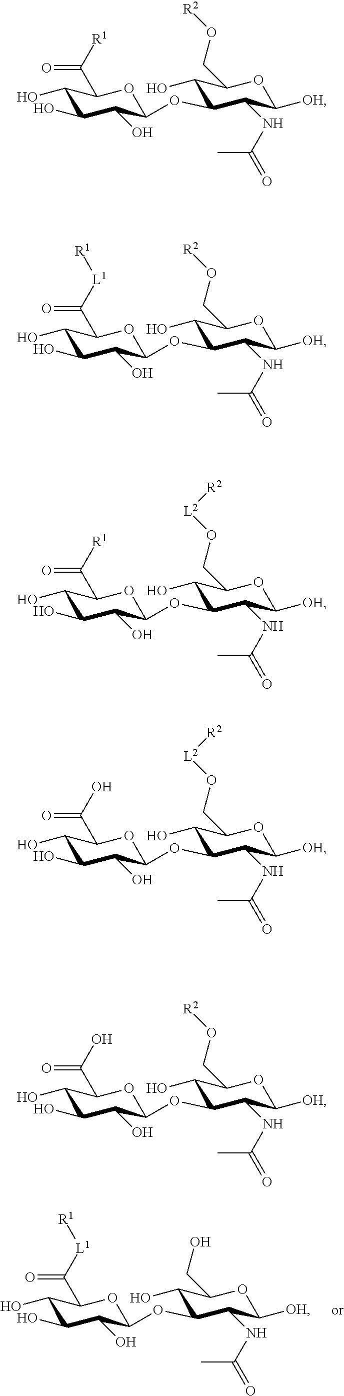

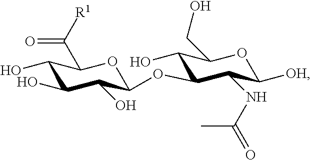

where are least one of

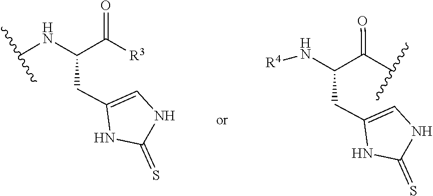

is present. In various examples where a compound has an R¹, at least a portion of the R¹ groups (e.g., at least one) are

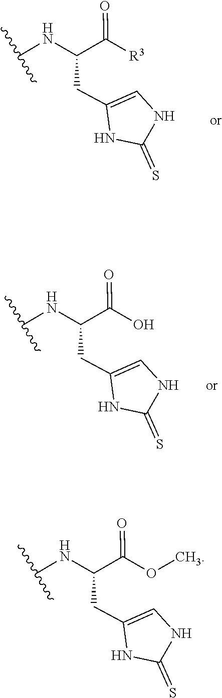

In various examples, the compound has the following structure:

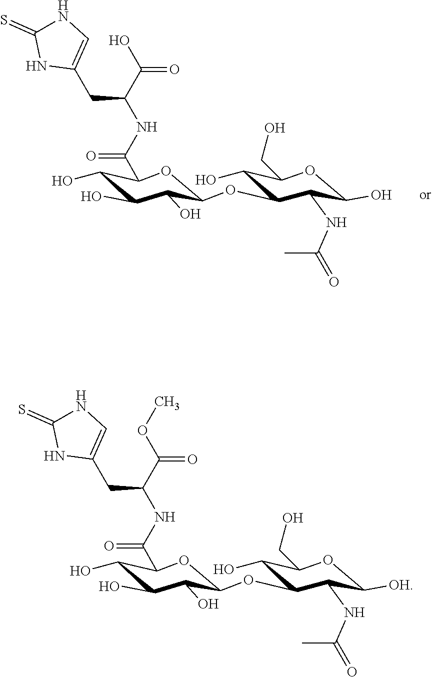

In an aspect, the present disclosure provides compositions. The composition may comprise a polymeric compound and/or compound of the present disclosure. The composition may also comprise a pharmaceutically acceptable carrier.

The composition can comprise a polymeric compound and/or compound in a pharmaceutically acceptable carrier (e.g., carrier). The carrier can be an aqueous carrier suitable for administration to individuals including humans. The carrier can be sterile. The carrier can be a physiological buffer. Examples of suitable carriers include sucrose, dextrose, saline, and/or a pH buffering element (such as, a buffering element that buffers to, for example, a pH from pH 5 to 9, from pH 6 to 8, (e.g., 6.5)) such as histidine, citrate, or phosphate. Additionally, pharmaceutically acceptable carriers may be determined in part by the particular composition being administered. Accordingly, there are a wide variety of suitable formulations of pharmaceutical compositions of the present disclosure. Additional, non-limiting examples of carriers include solutions, suspensions, and emulsions that are dissolved or suspended in a solvent before use, and the like. The composition may comprise one or more diluents. Examples of diluents, include, but are not limited to distilled water, physiological saline, vegetable oil, alcohol, dimethyl sulfoxide, and the like, and combinations thereof. Compositions may contain stabilizers, solubilizers, suspending agents, emulsifiers, soothing agents, buffers, preservatives, and the like, and combinations thereof. Compositions may be sterilized or prepared by sterile procedure. A composition of the disclosure may also be formulated into a sterile solid preparation, for example, by freeze-drying, and may be used after sterilization or dissolution in sterile injectable water or other sterile diluent(s) immediately before use. Additional examples of pharmaceutically acceptable carriers include, but are not limited to, sugars, such as, for example, lactose, glucose, and sucrose; starches, such as, for example, corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as, for example, peanut oil, cottonseed oil, safflower oil, sesame oil, olive oil, corn oil, and soybean oil; glycols, such as, for example, propylene glycol; polyols, such as, for example glycerin, sorbitol, mannitol, and polyethylene glycol; esters, such as, for example, ethyl oleate and ethyl laurate; agar; buffering agents, such as, for example, magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Additional non-limiting examples of pharmaceutically acceptable carriers can be found in: Remington: The Science and Practice of Pharmacy (2012) 22nd Edition, Philadelphia, PA. Lippincott Williams & Wilkins. For example, a composition comprises a modified peptide, and a sterile, suitable carrier for administration to individuals including humans—such as a physiological buffer such as sucrose, dextrose, saline, pH buffering (such as from pH 5 to 9, from pH 7 to 8, from pH 7.2 to 7.6, (e.g., 7.4)) element such as, for example, histidine, citrate, or phosphate. In various examples, the composition may be suitable for injection. Parenteral administration includes infusions and injections, such as, for example, intramuscular, intravenous, intraarterial, intraperitoneal, subcutaneous administration, and the like.

The compositions may be administered systemically. Compositions may be administered orally, may be administered parenterally, and/or intravenously. Compositions suitable for parenteral, administration may include aqueous and/or non-aqueous carriers and diluents, such as, for example, sterile injection solutions. Sterile injection solutions may contain anti-oxidants, buffers, bacteriostatic agents and solutes, which render the composition isotonic with the blood of the intended recipient. Aqueous and/or non-aqueous sterile suspensions may include suspending agents and thickening agents.

The compositions of the present disclosure may be administered systemically. The term “systemic” as used herein includes parenteral, topical, oral, spray inhalation, rectal, nasal, and buccal administration. The term “parenteral” as used herein includes subcutaneous, intravenous, intramuscular, intra-articular, intra-synovial, intrasternal, intrathecal, intrahepatic, intralesional, and intracranial administration. Preferably, the compositions are administered orally, intraperitoneally, or intravenously.

Examples of compositions include, but are not limited to, liquid solutions, such as, for example, an effective amount of a compound of the present disclosure suspended in diluents, such as, for example, water, saline or PEG 400. The liquid solutions described above may be sterile solutions. The compositions may comprise, for example, one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers.

The composition may further comprise compounds and/or polymeric compounds without 2-thioHis. For example, the composition may comprise:

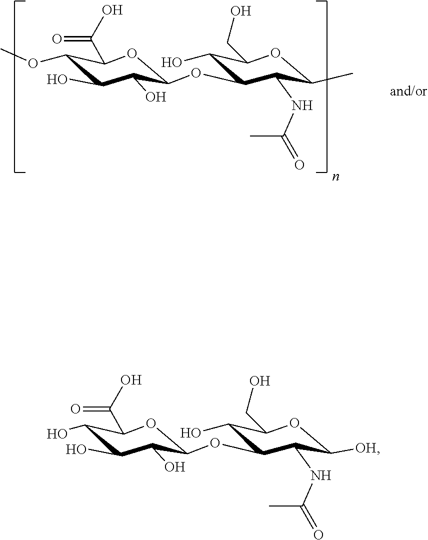

wherein n is 2 to 50, including all integer values and ranges therebetween. In various embodiments, such compositions comprise 0.1 to 99.9 mol % compounds and/or polymeric compounds comprising a 2-thioHis group.

In various embodiments, the composition may be a cosmetic formulation. The cosmetic formulation may be a skin care product. The skin care product may be a lotion, a cream, a dermal filler, an ointment, a paste, a foam, or the like. The skin care product may be suitable for topical application to an individual in need of treatment.

The composition may comprise one or more cosmetic components. Examples of cosmetic components include, but are not limited to, scented ingredients (e.g., essential oils and the like), exfoliating agents (e.g., salicylic acid and the like), lubricants (e.g., hyaluronic acid and the like), anti-cellulite agents (e.g., caffeine and the like), and the like, and various combinations thereof. Other examples of any of the foregoing components are known in the art and may be suitable for use in the composition of the present disclosure.

Addition ingredients may also optionally be included in the cosmetic compositions. Non-limiting examples include water, non-volatile fatty substances, inorganic pigments, soft focus particles/powders, fragrances, preservatives, coalescents, wetting agents, water-soluble solvents, emollients, suspending agents, surfactants, actives, and the like. One unique feature of the cosmetic compositions is that they may include water (aqueous compositions) or may be free of water (anhydrous compositions).

The compositions may be suitable for topical or transdermal application or oral administration. The compositions can be produced in any solid, liquid or semi-solid formulation, including creams, emulsions, anhydrous compositions, aqueous dispersions, oils, foams, lotions, gels, ointments, sprays or aerosols or any other form suitable via the skin or mucosal surface. The formulations can be incorporated into support materials that can be applied to a wound surface, such as, for example, bandages, gauzes, clothing, diapers, dressings, adhesive or non-adhesive patches, and the like. The formulations can also be incorporated into cosmetic materials, such as foundations, lipsticks, moisturizers, creams, masks, and the like.

In an aspect, the present disclosure provides methods of using polymeric compounds and/or compounds of the present disclosure of the present disclosure. The method may be a method for quenching free radicals or reducing oxidative stress via reduction of the thiohistindyl group.

For example, a method may comprise administering and/or contacting a therapeutically effective amount of a polymeric compound and/or compound of the present disclosure or composition comprising the polymeric compound and/or compound of the present disclosure to an individual in need of treatment of oxidative stress or for quenching free radicals in the individual. The administration may result in decreasing the amounts of free radicals, thereby reducing the oxidative stress on the individual. In various example, the free radical is singlet oxygen. Following contact with the polymeric compound and/or compound of the present disclosure, sulfate will be produced.

The phrase “therapeutically effective amount” is used herein to mean an amount sufficient to reduce by at least about 15 percent, preferably by at least 50 percent, more preferably by at least 90 percent, and most preferably prevents oxidative stress in the individual. Alternatively, a therapeutically effective amount is sufficient to cause an improvement in a clinically significant condition in the host.

In a method of the present disclosure, compositions may be administered by various routes. The compositions of the present disclosure may be administered systemically, orally, or topically.

An individual in need of treatment may be a human or non-human mammal. Non-limiting examples of non-human mammals include cows, pigs, mice, rats, rabbits, cats, dogs, other agricultural animal, pet, service animals, and the like.

The steps of the method described in the various embodiments and examples disclosed herein are sufficient to carry out the methods of the present disclosure. Thus, in an embodiment, the method consists essentially of a combination of the steps of the methods disclosed herein. In another embodiment, the method consists of such steps.

The following Statements provide various embodiments of the present disclosure.

Statement 1. A polymeric compound having the following structure:

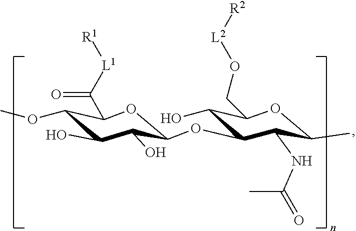

wherein
L¹ and L² are each optional and are linking groups;

R¹ is OH or

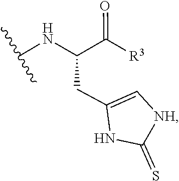

wherein R³ is OH, OCH₃, L¹, L¹-R³, or

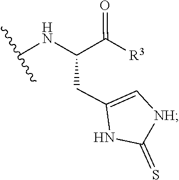

R² is H or

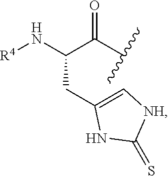

wherein R⁴ is H, L², L²-R⁴, or

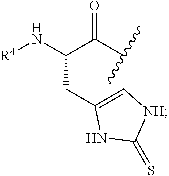

n is 2 to 50, including all integer values and ranges therebetween; and
   wherein at least one

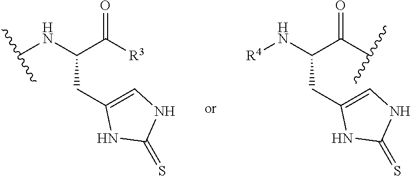

is present.

Statement 2. A polymeric compound according to Statement 1, wherein the linking groups are independently chosen from amino acid groups, peptides groups, PEG groups, diethylamine groups, ethyl disulfide groups, cysteamine groups, and the like, and combinations thereof.

Statement 3. A polymeric compound according to Statement 2, wherein the amino acid groups are glycyl groups or prolyl group.

Statement 4. A polymer compound according to Statement 1, wherein there is no L¹ and/or L².

Statement 5. A polymeric compound according to any one of the preceding Statements, wherein there is no L² and at least a portion of the R² groups are H.

Statement 6. A polymeric compound according to Statement 5, wherein the polymeric compound has the following structure:

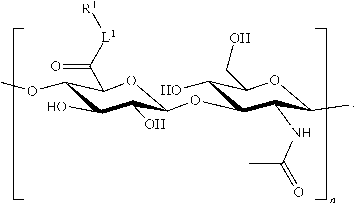

Statement 7. A polymeric compound according to any one of Statements 1-5, wherein there is no L and at least a portion of the R¹ groups are:

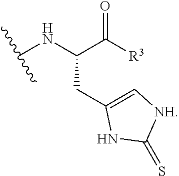

Statement 8. A polymeric compound according to any one of Statements 1-5, wherein there is no L¹ and at least a portion of the R¹ groups are:

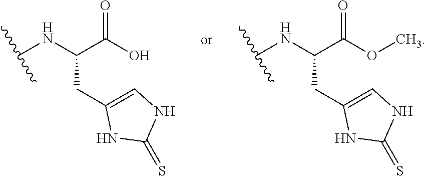 [SMILES: *N[C@@H](Cc1c[nH]c(=S)[nH]1)C(=O)O ; "or" ; *N[C@@H](Cc1c[nH]c(=S)[nH]1)C(=O)OC]

Statement 9. The polymeric compound according to Statement 8, wherein the polymeric compound has the following structure:

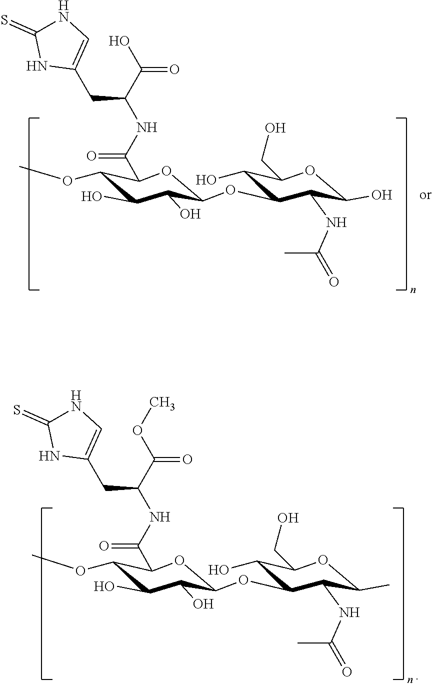

Statement 10. A composition comprising a plurality of polymers, wherein at least a portion of the polymers are the polymeric compounds according to any one of the preceding Statements.

Statement 11. A composition according to Statement 10, wherein the composition further comprises polymers having the following structure:

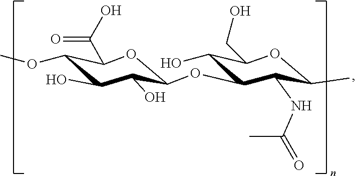

wherein n is 2 to 50, including all integer values and ranges therebetween. Statement 12. A composition according to Statement 11, wherein 0.1 to 99.9 mol % of the total amount of polymer is the polymeric compound according to any one of claims 1-8, including all 0.1 mol % values and ranges therebetween.

Statement 13. A composition according to any one of Statements 10-12, further comprising a pharmaceutically acceptable carrier.

Statement 14. A cosmetic formulation comprising a composition according to any one of Statements 10-13.

Statement 15. A cosmetic formulation according to Statement 14, wherein the cosmetic is a skin care product.

Statement 16. A cosmetic formulation according to Statement 15, wherein the skin care product is a lotion, a cream, a dermal filler, an ointment, a paste, or a foam.

Statement 17. A compound having the following structure:

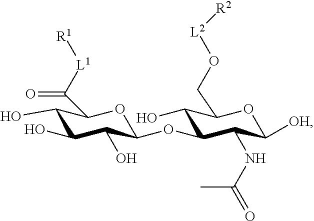

wherein
L¹ and L² are each optional and are linking groups;
R¹ is OH or

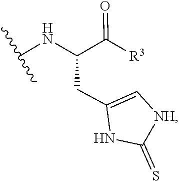

wherein R³ is OH, OCH³, L¹, L¹-R³, or

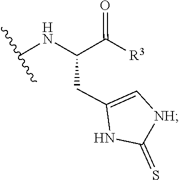

R² is H or

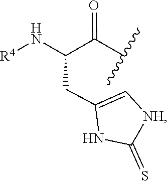

wherein R⁴ is H, L², L²-R⁴, or

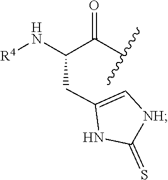

and
   wherein at least one of

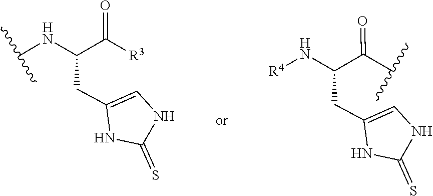

are present.

Statement 18. A compound according to Statement 17, wherein the linking groups are independently chosen from amino acid groups, peptides groups, PEG groups, diethylamine groups, ethyl disulfide groups, cysteamine groups, and the like, and combinations thereof. Statement 19. A compound according to Statement 18, wherein the amino acid groups are glycyl groups or prolyl group.

Statement 20. A compound according to Statement 17, wherein there is no L¹ and/or L².

Statement 21. A compound according to any one of Statements 17-20, wherein there is no L² and R² is H.

Statement 22. A compound according to Statement 21, wherein the compound has the following structure:

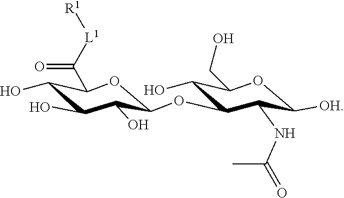

Statement 23. A compound according to any one of Statements 17-22, wherein there is no L¹ and R¹ is:

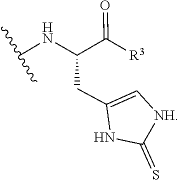

Statement 24. A compound according to any one of Statements 17-23, wherein there is no L¹ and R¹ groups is:

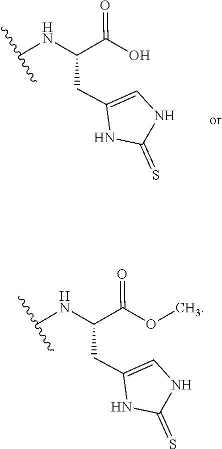 [SMILES: *N[C@@H](Cc1c[nH]c(=S)[nH]1)C(=O)O ; *N[C@@H](Cc1c[nH]c(=S)[nH]1)C(=O)OC]

Statement 25. A compound according to Statement 24, wherein the polymeric compound has the following structure:

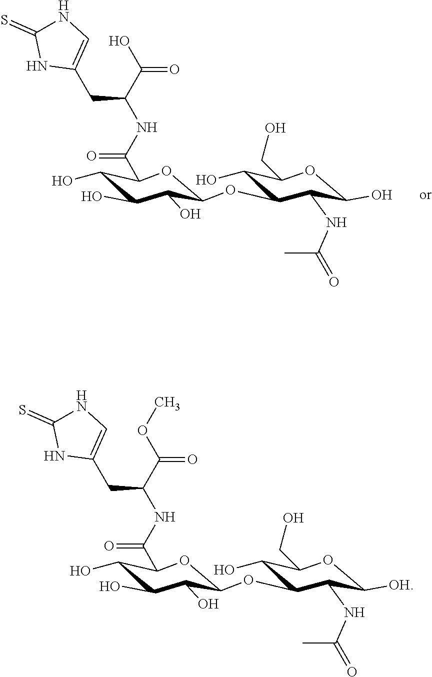

Statement 26. A composition comprising a plurality of compounds, wherein at least a portion of the compounds are compounds according to any one of Statements 17-25.

Statement 27. A composition according to Statement 26, wherein the composition further comprises compounds having the following structure:

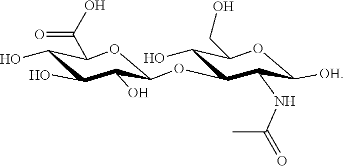

Statement 28. A composition according to Statement 26 or Statement 27, wherein 0.1 to 99.9 mol % of the total amount of compounds are compounds according to any one of Statements 17-25, including all 0.1 mol % values and ranges therebetween.

Statement 29. A composition according to any one of Statements 26-28, further comprising a pharmaceutically acceptable carrier.

Statement 30. A cosmetic formulation comprising a composition according to any one of Statements 26-29.

Statement 31. A cosmetic formulation according to Statement 30, wherein the cosmetic is a skin care product.

Statement 32. A cosmetic formulation according to claim 31, wherein the skin care product is a lotion, a cream, a dermal filler, an ointment, a paste, or a foam.

Statement 33. A composition comprising polymeric compounds according to any one of Statements 1-9 and/or a compounds according to any one of Statements 17-25.

Statement 34. A composition according to Statement 33, wherein the composition further comprises polymers having the following structure:

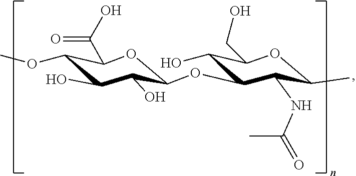

wherein n is 2 to 50, including all integer values and ranges therebetween and/or

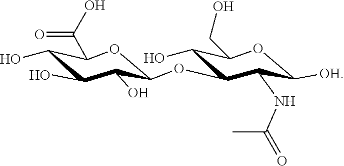

Statement 35. A composition according to Statements 33 or 34, further comprising a pharmaceutically acceptable carrier.

Statement 36. A cosmetic formulation comprising a composition according to any one of Statements 33-35.

Statement 37. A cosmetic formulation according to Statement 36, wherein the cosmetic is a skin care product.

Statement 38. A cosmetic formulation according to Statement 37, wherein the skin care product is a lotion, a cream, a dermal filler, an ointment, a paste, or a foam.

Statement 39. A method for quenching free radicals in an individual comprising:
contacting the individual with the composition according to any one of Statements 10-13, 26-29, or 33-35 and/or a cosmetic according to any one of Statements 14-16, 30-32, and 36-38
   wherein the contacting results in reduction of a thiohistidinyl group.

The following example is presented to illustrate the present disclosure. It is not intended to be limiting in any matter.

### EXAMPLE

As discussed herein, hyaluronic acid is already added to billions of dollars' worth of cosmetics each year. The polymer and/or monomers of the present disclosure should function the same way as the unmodified hyaluronic acid, but have added antioxidant properties. Specifically, it will be able to act as a “photoprotectant,” protecting the skin from photoaging since 2-thiohistidine quenches singlet oxygen. Singlet oxygen is a form of excited oxygen that forms when sunlight reacts with endogenous photosensitizers in the skin. Singlet oxygen is a strong oxidant that oxidizes protein, nucleic acids, and lipids in the skin and is one cause of photoaging. In addition to its properties as a photoprotectant, the polymer and/or monomers of the present disclosure will have further antioxidant properties as it will quench free radicals in the skin. The oxidized form of 2-thiohistidine that results from the reaction with a free radical can be recycled back to the original form by endogenous antioxidants present in the skin such as glutathione and ascorbate. Another advantage that the polymer and/or monomers of the present disclosure has is that if recycling back to the original form does not occur when it reacts with a free radical, the conjugate will detoxify the free radical with release of nontoxic sulfate. The 2-thiohistidine will be converted to histidine, a naturally occurring amino acid, also a non-toxic by product. Another significant advantage that The polymer and/or monomers of the present disclosure will have over preparations that contain a mixture of hyaluronic acid and ergothioneine is that in such preparations, the ergothioneine is free to diffuse away from the site of application. The polymer and/or monomers of the present disclosure has a covalent bond to hyaluronic acid, a high molecular weight polymer, it is forced to stay at the site of application, which is a significant advantage over simple addition of ergothioneine to a cosmetic product.

Although the present disclosure has been described with respect to one or more particular embodiments and/or examples, it will be understood that other embodiments and/or examples of the present disclosure may be made without departing from the scope of the present disclosure.

1. A polymeric compound having the following structure: 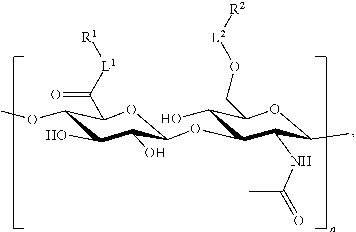
wherein
L¹ and L² are each optional and are linking groups;
R¹ is OH or 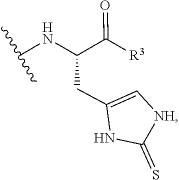
wherein R³ is OH, OCH₃, L¹, L¹-R³, or 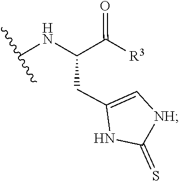
R² is H or 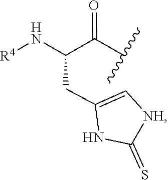
wherein R⁴ is H, L², L²-R⁴, or 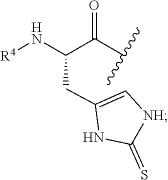
n is 2 to 50, including all integer values and ranges therebetween; and
wherein at least one 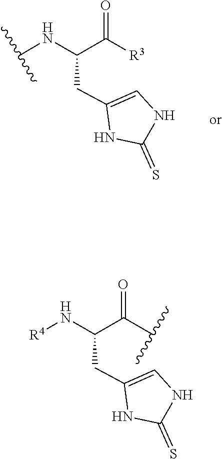
is present.

2. The polymeric compound according to claim 1, wherein the linking groups are independently chosen from amino acid groups, peptides groups, PEG groups, diethylamine groups, ethyl disulfide groups, cysteamine groups, and combinations thereof.

3. The polymeric compound according to claim 2, wherein the amino acid groups are glycyl groups or prolyl group.

4. The polymer compound according to claim 1, wherein there is no L¹ and/or L².

5. The polymeric compound according to claim 1, wherein there is no L² and at least a portion of the R² groups are H.

6. The polymeric compound according to claim 5, wherein the polymeric compound has the following structure:

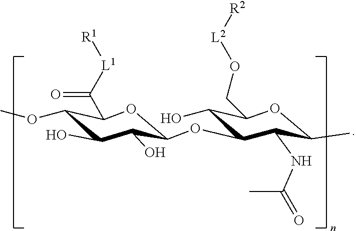

7. The polymeric compound according to claim 1, wherein there is no L¹ and at least a portion of the R¹ groups are:

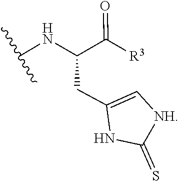

8. The polymeric compound according to claim 1, wherein there is no L¹ and at least a portion of the R¹ groups are:

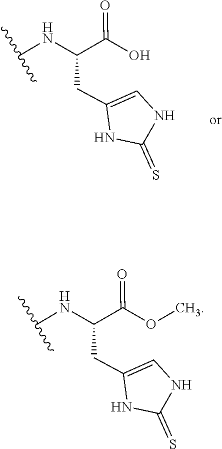 [SMILES: *N[C@@H](Cc1c[nH]c(=S)[nH]1)C(=O)O ; *N[C@@H](Cc1c[nH]c(=S)[nH]1)C(=O)OC]

9. The polymeric compound according to claim 8, wherein the polymeric compound has the following structure:

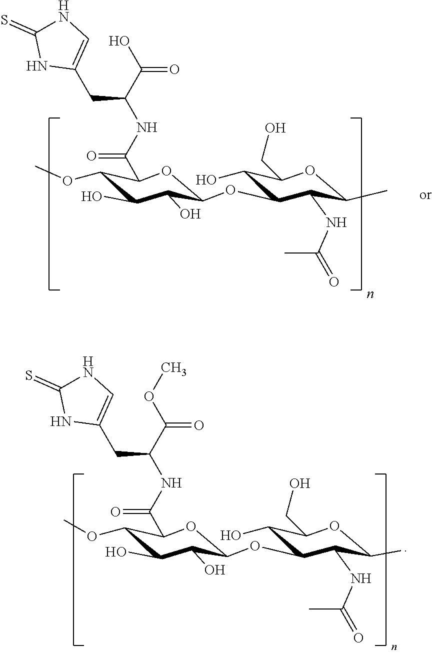

10. A composition comprising a plurality of polymers, wherein at least a portion of the polymers are the polymeric compounds according to claim 1.

11. The composition according to claim 10, wherein the composition further comprises polymers having the following structure: 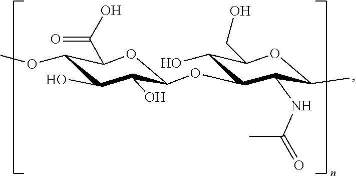
wherein n is 2 to 50.

12. The composition according to claim 11, wherein 0.1 to 99.9 mol % of the total amount of polymer is the polymeric compounds.

13. The composition according to claim 10, further comprising a pharmaceutically acceptable carrier.

14. A cosmetic formulation comprising a composition according to claim 10.

15. The cosmetic formulation according to claim 14, wherein the cosmetic is a skin care product.

16. The cosmetic formulation according to claim 15, wherein the skin care product is a lotion, a cream, a dermal filler, an ointment, a paste, or a foam.

17. A compound having the following structure: 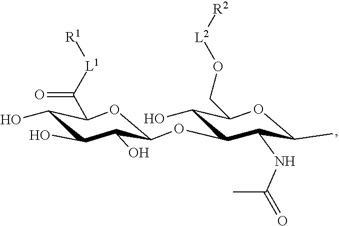
wherein
L¹ and L² are each optional and are linking groups;
R¹ is OH or 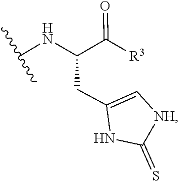
wherein R³ is OH, OCH₃, L¹, L¹-R³, or 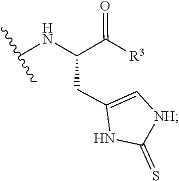
R² is H or 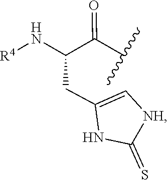
wherein R⁴ is H, L², L²-R⁴, or 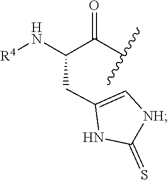
and
wherein at least one of 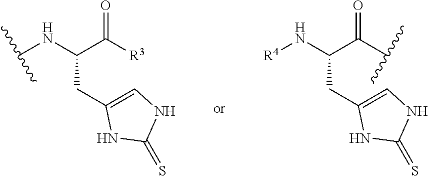
are present.

18. The compound according to claim 17, wherein the linking groups are independently chosen from amino acid groups, peptides groups, PEG groups, diethylamine groups, ethyl disulfide groups, cysteamine groups, and the like, and combinations thereof.

19. The compound according to claim 18, wherein the amino acid groups are glycyl groups or prolyl groups.

20. The compound according to claim 17, wherein there is no L¹ and/or L².

21. The compound according to claim 17, wherein there is no L² and R² is H.

22. The compound according to claim 21, wherein the compound has the following structure:

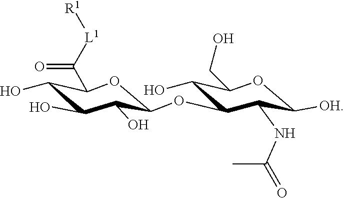

23. The compound according to claim 17, wherein there is no L¹ and R¹ is:

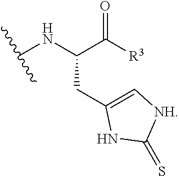

24. The compound according to 17, wherein there is no L¹ and R¹ groups is:

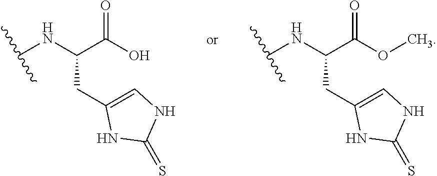 [SMILES: *N[C@@H](Cc1c[nH]c(=S)[nH]1)C(=O)O ; "or" ; *N[C@@H](Cc1c[nH]c(=S)[nH]1)C(=O)OC]

25. The compound according to claim 24, wherein the polymeric compound has the following structure:

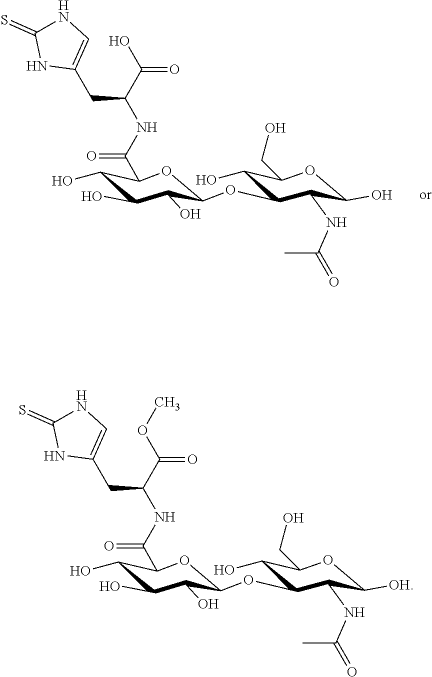

26. A composition comprising a plurality of compounds, wherein at least a portion of the compounds are compounds according to claim 17.

27. The composition according to claim 26, wherein the composition further comprises compounds having the following structure:

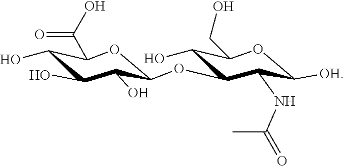

28. The composition according to claim 26, wherein 0.1 to 99.9 mol % of the total amount of compounds are compounds having the following structure:

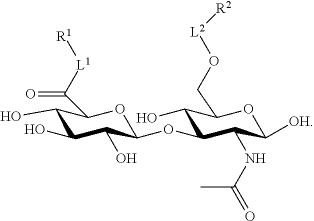

29. The composition according to claim 26, further comprising a pharmaceutically acceptable carrier.

30. A cosmetic formulation comprising a composition according to claim 26.

31. The cosmetic formulation according to claim 30, wherein the cosmetic is a skin care product.

32. The cosmetic formulation according to claim 31, wherein the skin care product is a lotion, a cream, a dermal filler, an ointment, a paste, or a foam.

33. A composition comprising one or more polymeric compounds having the following structure: 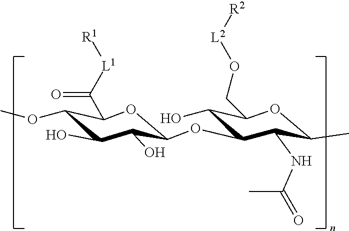
and/or one or more compounds having 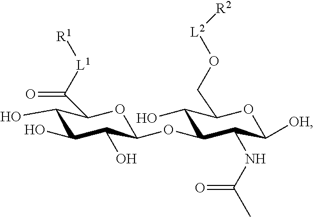
wherein
L¹ and L² are each optional and are linking groups;
R¹ is OH or 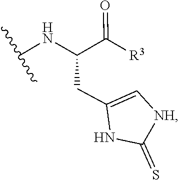
wherein R³ is OH, OCH₃, L¹, L¹-R³, or 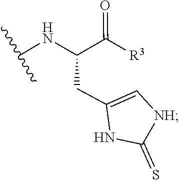
R² is H or 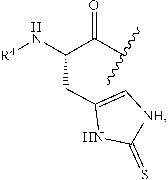
wherein R⁴ is H, L², L²-R⁴, or 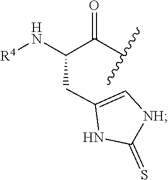
and
wherein at least one of 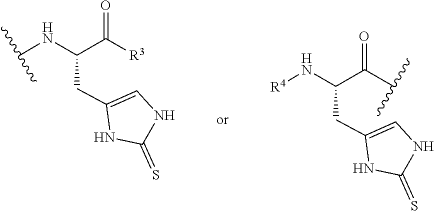
are present on at least one polymeric compound and/or compound.

34. The composition according to claim 33, wherein the composition further comprises polymers having the following structure: 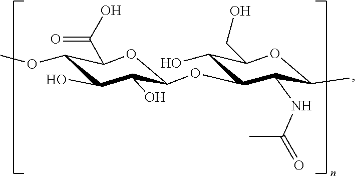
wherein n is 2 to 50, and/or

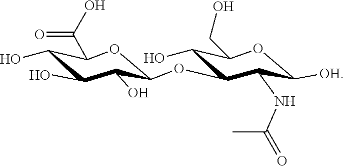

35. The composition according to claim 33, further comprising a pharmaceutically acceptable carrier.

36. A cosmetic formulation comprising a composition according claim 33.

37. The cosmetic formulation according to claim 36, wherein the cosmetic is a skin care product.

38. The cosmetic formulation according to claim 37, wherein the skin care product is a lotion, a cream, a dermal filler, an ointment, a paste, or a foam.

39. A method for quenching free radicals in an individual comprising:
contacting the individual with the composition and/or cosmetic comprising one or more polymeric compounds having the following structure: 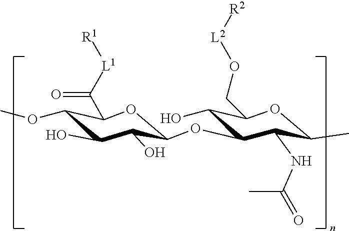
and/or one or more compounds having 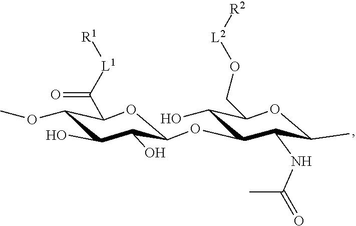
wherein
L¹ and L² are each optional and are linking groups;
R¹ is OH or 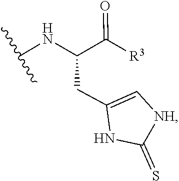
wherein R³ is OH, OCH₃, L¹, L¹-R³, or 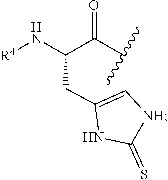
R² is H or 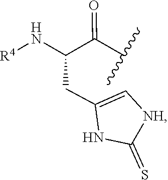
wherein R⁴ is H, L², L²-R⁴, or 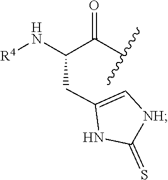
and
wherein at least one of 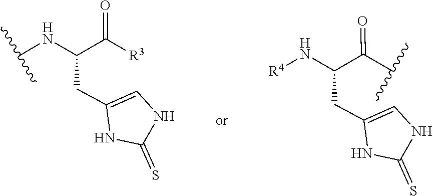
are present on at least one polymeric compound and/or compound, wherein the contacting results in the chemical reduction of a thiohistidinyl group.
